SEQUENCE LISTING (4 sequences)

SEQ ID NO: 1 - DNA, 21 bp, Artificial Sequence
  MMP-1 forward primer
ccccaaaagcgtgtgacagta

SEQ ID NO: 2 - DNA, 19 bp, Artificial Sequence
  MMP-1 reverse primer
ggtagaagggatttgtgcg

SEQ ID NO: 3 - DNA, 20 bp, Artificial Sequence
  GAPDH forward primer
gagtcaacggatttggtcgt

SEQ ID NO: 4 - DNA, 20 bp, Artificial Sequence
  GAPDH reverse primer
ttgattttggagggatctcg

(19) United States
(12) Patent Application Publication
(10) Pub. No.: US 2019/0021976 A1
(43) Pub. Date: Jan. 24, 2019

(54) COMPOSITION FOR PREVENTING, IMPROVING OR TREATING PHOTOAGING OF SKIN COMPRISING DHPV

(71) Applicants: SUNGKWANG MEDICAL FOUNDATION, SEOUL (KR); College of Medicine Pochon CHA University Industry-Academic Cooperation Foundation, Pocheon-si (KR)

(72) Inventors: David Tae Aug KIM, Seoul (KR); Dong Hyun Kim, Seoul (KR); A-Ram Kim, Goyang-si (KR)

(21) Appl. No.: 15/809,042

(22) Filed: Nov. 10, 2017

(30) Foreign Application Priority Data
Dec. 30, 2016 (KR) 10-2016-0184101

(51) Int. Cl. A61K 8/49; A61Q 19/08

(52) U.S. Cl. CPC A61K 8/4973; A61Q 19/08

(57) ABSTRACT

The present invention relates to a photoaging inhibitor, and more specifically it relates to a pharmaceutical composition, a food composition or a cosmetic composition for preventing, improving or treating photoaging of skin, comprising DHPV (5-(3′,4′-Dihydroxyphenyl)-Γ-valerolactone) which is a metabolite of cacao. Since DHPV, an active ingredient comprised in the composition according to the present invention is a metabolite of cacao which is a botanical raw material harmless to human body and is not only excellent in biosafety, but also regulates expression of proteins related to skin aging epigenetically, thereby exhibiting an excellent skin aging-preventing function at a low concentration of 1 nM based on skin cells, the composition comprising the same is excellent in use as medicines, cosmetics and foods for preventing, improving and treating skin aging.

### TECHNICAL FIELD

The present invention relates to a photoaging inhibitor, and more specifically it relates to a pharmaceutical composition, a food composition or a cosmetic composition for preventing, improving or treating photoaging of skin, comprising DHPV (5-(3′,4′-Dihydroxyphenyl)-Γ-valerolactone) which is a metabolite of cacao.

### BACKGROUND ART

As the level of living increases, there is a growing interest in preventing skin damage caused by exposure to sunlight as the desire to maintain beautiful and healthy skin is increased and outdoor activities are increasing, and studies on inhibition of skin aging are under progress. The phenomenon of skin damage such as skin elasticity and moisture decrease and wrinkles are formed, etc. when repeated or long-term exposure to ultraviolet radiation (UVR) of sunlight is called photoaging. The ultraviolet radiation is classified to UVA (ultraviolet A, 320-400 nm), UVB (ultraviolet B, 280-320 nm), UVC (ultraviolet C, 200-280 nm) by wavelength. Among them, UVB is very harmful light, and it relates to epidermis damage or causes skin erythema, pigmentation, acanthosis, etc. Also, it causes damage to lipids, proteins, enzymes, etc. which make up skin through induction of unbalance of antioxidant mechanism, thereby deepening inflammation reaction, skin cancer, and photoaging. On the other hand, UVA is less powerful than UVB for a short period of time, but its wavelength is so long that it penetrates deeper into the skin and damages dermis of skin, thereby blackening the skin and promoting skin aging. Therefore, the efficacy standards of materials and products which can inhibit UVA and UVB are used as UVR barrier index. ROS (reactive oxygen species) whose production is induced by UVB exposure is one of causes of photoaging, and it stimulates intracellular signal transduction system (Wu et al., Proteomic analysis of UVB-induced protein expression- and redox-dependent changes in skin fibroblasts using lysine- and cysteine-labeling two-dimensional difference gel electrophoresis. J Proteomics 75, 2012, 1991-2014) and causes damage of skin tissue by inducing oxidative stress on biomolecules such as DNA, lipids and proteins. ROS activates transcription factors of Ap-1 (activator protein-1) and NF-κB (nuclear factor kappa-light-chain-enhancer of activator B cells) by stimulation of epidermal keratinocytes and dermal fibroblasts, and secretes inflammation-related cytokine, thereby increasing expression of MMPs (matrix metalloproteinase) and promoting degradation of collagen and elastic fibers constituting skin tissue to form wrinkles (Darr et al., Free radicals in cutaneous biology. J Invest Dermatol 102, 1994, 671-675). In vivo, antioxidant enzymes such as CAT (catalase), SOD (superoxide dismutase) and GST (glutathione-s-transferase), etc. act to protect against lipid peroxides and oxidases generated and activated from such ROS stimulation (Hassan H M, Biosynthesis and regulation of superoxide dismutases. Free Radic Biol Med 5,1988, 377-384), and in vitro, physiologically active substances such as vitamin C, E and polyphenols and flavonoids have been reported to protect against damage caused by ROS produced (Borek et al., Dietary antioxidants and human cancer. Integr Cancer Ther 3, 2004, 333-341). As the role and importance of antioxidants for skin damage and wrinkle improvement due to photoaging of skin have been emphasized, studies have been actively carried out to develop natural functional materials and products that have antioxidant activity and exhibit efficacy without side effects through long-term use.

In order to prevent skin aging, cosmetics containing various animals, plants, microorganisms and physiologically active substances obtained by organic synthesis have been used, and in general, a method of applying an AHA, retinoic acid, retinol, ascorbic acid, etc. and an herb extract containing antioxidant ingredient, polyphenol is mainly used. In Korea, functional cosmetics are classified into three types of wrinkle improvement, whitening and UVR protection, and products for improving wrinkle prevention among them are the most popular and relatively high priced products are the mainstream. Recently, due to knowledge of human harmfulness of compounds and components of animal-derived raw materials, regulations for use of harmful raw materials for animal have become stronger, and the interest in the plant-derived raw materials harmless to human bodies is increasing.

Meanwhile, cacao (Theobroma cacao) is a branch of the dicotyledonous plant columniferae Sterculiaceae and the origin of the American tropical region. Cacao bean contains a large amount of polyphenols, and in particular, due to Cacao bean (CB)-derived polyphenols such as flavan-3-ol like epicatechin, procyanidin, catechin polymer, etc. which are known to be stored in a pigment cell of cotyledon in an amount of about 10% Cacao bean weight, it has been well known that Cacao bean has a much higher antioxidant potential than green tea or red wine. Furthermore, recent studies have shown that such antioxidant action can improve skin conditions.

Under these circumstances, the present inventors have conducted intense studies to develop an effective and harmless plant-derived material for preventing skin aging, especially photoaging, and demonstrated that DHPV, one of the metabolites of cacao, inhibits expression of proteins specific to skin aging epigenetically and exhibits an effect of preventing skin aging, especially photoaging, thereby completing the present invention.

### DISCLOSURE

### Technical Problem

Therefore, a purpose of the present invention is to provide a pharmaceutical or cosmetic composition for preventing, improving or treating photoaging of skin, comprising DHPV (5-(3′,4′-Dihydroxyphenyl)-Γ-valerolactone) which is a metabolite of cacao as an active ingredient.

Another purpose of the present invention is to provide a pharmaceutical composition for promoting skin elasticity and improving wrinkles, comprising DHPV (5-(3′,4′-Dihydroxyphenyl)-Γ-valerolactone) as an active ingredient.

### Technical Solution

As one aspect to solve the above problem, the present invention relates to a composition for preventing, improving or treating photoaging of skin, comprising DHPV (5-(3′,4′-Dihydroxyphenyl)-r-valerolactone) of the following chemical formula 1 as an active ingredient.

The above composition may be a pharmaceutical, cosmetic or food composition.

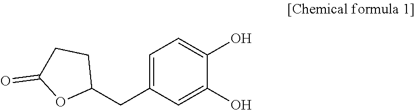 [SMILES: O=C1CCC(Cc2ccc(O)c(O)c2)O1]

DHPV of the chemical formula 1 is a phenolic compound of cacao, and it may be obtained from cacao, and specifically it is a major metabolite of cacao and it may be prepared by publicly known methods from cacao.

As above, there are various prior arts concerning the antioxidative effect on cacao, etc., but there has been no report on prevention, improvement or treatment action of photoaging of skin of cacao metabolites, especially DHPV. Specifically, DHPV regulates expression changes of a skin photoaging-related epigenetic gene, DNA methylation, thereby preventing, improving or treating photoaging of skin and related skin symptoms or diseases. As one desirable aspect, DHPV regulates expression level of MMP1 and involucrine (IVL) which are known as a maker of photoaging of skin. The concentration of IVL in skin cells exposed to UVB is significantly increased. DHPV of the present invention epigenetically reduces expression level of increased skin aging specific proteins, preferably IVL and PFN1 proteins, in a manner similar to that of a cell not exposed to UVB. Thereby, the present invention can provide an excellent effect of prevention, improvement and treatment in improving photoaging of skin by DHPV which is a metabolite derived from a botanical raw material.

As one desirable aspect, the present invention is a pharmaceutical composition for preventing or treating photoaging of skin comprising DHPV (5-3′,4′-Dihydroxyphenyl)-Γ-valerolactone) of the chemical formula 1.

In case that the composition according to the present invention is a pharmaceutical composition, the content of DHPV as an active ingredient in the composition may be appropriately controlled by type and purpose of use, patient condition, type and severity of symptoms, etc., and it may be 0.001 to 99.9% by weight, or 0.1 to 99.9% by weight, preferably 0.1 to 50% by weight or 0.1 to 40% by weight, based on the total composition weight. However, it is not limited to the above range, because may be increased or decreased depending on needs of a medicinal person, and it may be appropriately increased or decreased according to various factors such as diet, nutritional status, damage degree of cartilage or joint, degree of progression of the above diseases, etc.

The composition according to the present invention may be administered to mammals including humans by a variety of routes. The administration manner may be any manner conventionally used, and for example, the composition may be administered by oral or parenteral (for example, skin, intravenous, intramuscular, subcutaneous) route, etc., and preferably it may be administered orally, but it may be administered in the form of a parenteral formulation such as an ointment which can be applied directly to the skin area where photoaging is a concern, in order to enhance an effect of preventing or treating photoaging of skin. In particular, DHPV can be formulated as an ointment to be applied to skin because of its high skin permeability and can be suitably used. The composition of the present invention may be formulated into oral formulations such as powders, granules, tablets, capsules, solutions, suspensions, emulsions, syrups, aerosols, etc. or parenteral formulations such as epidermal formulations, suppositories, ointments and sterile injection solutions, etc., according to conventional methods, respectively and used.

The composition of the present invention may further contain pharmaceutically acceptable and physiologically acceptable carriers, adjuvants such as excipients and diluents, etc. For example, in case of oral formulations, formulations may be prepared using excipients, binders, disintegrating agnets, lubricants, solubilizers, suspending agents, preservatives or extenders, etc.

The dose of the pharmaceutical composition of the present invention may be determined by a specialist according to various factors such as patient condition, age, body weight, damage degree of cartilage, degree of progression of disease, etc. In addition, the daily dose of the pharmaceutical composition, or a half, ⅓ or ¼ dose thereof may be contained per unit formulation, and the composition may be administered 1 to 6 times a day.

As another preferable aspect, the present invention relates to a food composition for preventing or improving photoaging of skin comprising DHPV (5-3′,4′-Dihydroxyphenyl)-Γ-valerolactone) of the chemical formula 1. The food is a health supplement food, a health functional food, a functional food, etc., but not limited thereto, and it is included that DHPV of the present invention is added to a natural food, a processed food, general food materials, etc. The food composition of the present invention may be used as it is or in combination with other food or food compositions, and may be suitable used according to conventional methods. The amount of active ingredients to be mixed may be appropriately determined depending on the intended use thereof. Generally, DHPV of the present invention may be added in an amount of 0.1 to 90 parts by weight, preferably 1 to 60 parts by weight, based on 100 parts by weight of the raw material for food or beverage in the production of food or beverage. The effective dose of DHPV may be used in accordance with the effective dose of the pharmaceutical composition, but may be less than the above range in case of long-term ingestion for prevention or improvement of photoaging of skin, and the active ingredient is a metabolite of cacao and may be used in an amount exceeding the above range because there is no problem in terms of safety. There is no particular limitation on the kind of the food. The food composition comprising DHPV of the above chemical formula 1 may be used in the form of food for oral administration such as tablets, hard or soft capsules, solutions, suspensions, and the like, and these formulations may be prepared using acceptable conventional carriers, for example, in case of oral formulations, excipients, binders, disintegrating agents, lubricants, solubilizers, suspending agents, preservatives or extenders, etc. Examples of the foods to which DHPV can be added include meats, sausages, breads, chocolates, candies, snacks, confectionery, pizza, ramen, other noodles, gums, dairy products including ice creams, many kinds of soups, beverages, teas, drinks, alcoholic beverages and vitamin complexes, other nutrients, etc., but not limited thereto.

As other preferable aspect, the present invention relates to a cosmetic composition for preventing or improving photoaging of skin comprising DHPV (5-3′,4′-Dihydroxyphenyl)-Γ-valerolactone) of the chemical formula 1. Specifically, DHPV which is comprised in the composition of the present invention may be appropriately used as a raw material of cosmetics which are applied to skin because of high skin permeability.

The formulation of the cosmetic composition is not particularly limited, and it may be appropriately selected according to purposes. For example, the formulation may be selected from the group consisting of skin lotion, skin softener, skin toner, astringent, lotion, milk lotion, moisture lotion, nutrition lotion, massage cream, nutrition cream, moisture cream, hand cream, foundation, essence, nutrition essence, pack, soap, cleansing foam, cleansing lotion, cleansing cream, body lotion and body cleanser, but not limited thereto. In case that the formulation of the present invention is paste, cream or gel, animal fiber, plant fiber, wax, paraffin, starch, tragacanth, cellulose derivative, polyethylene, glycol, silicone, bentonite, silica, talc or zinc oxide, etc. may be used as a carrier ingredient. In case that the formulation of the present invention is powder or spray, lactose, talc, silica, aluminum hydroxide, calcium silicate or polyamide powder may be used as a carrier ingredient, and particularly in case of spray, a propellant such as chlorofluorohydrocarbons, propane/butane or dimethyl ether may be additionally comprised. In case that the formulation of the present invention is solution or emulsion, solvent, solvation agent, or emulsifier may be used, and for example, there are water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or sorbitan fatty acid ester. In case that the formulation of the present invention is suspension, liquid diluent such as water, ethanol or propylene glycol, suspending agent such as ethoxylated isostearyl alcohol, polyoxyethylene sorbitol ester and polyoxyethylene sorbitan ester, microcrystalline cellulose, aluminum metahydroxide, bentonite, agar or tragacanth, etc. may be used. In case that the formulation of the present invention is surface-active agent-containing cleansing, aliphatic alcohol sulfate, aliphatic alcohol ether sulfate, sulfosuccinic acid monoester, isethionate, imidazolinium derivative, methyltaurate, sarcosinate, fatty acid amide ether sulfate, alkylamidobetaine, aliphatic alcohol, fatty acid glyceride, fatty acid diethanolamide, vegetable oil, linolenic derivative, or ethoxylated glycerol fatty acid ester, etc. may be used. The dose of the active ingredient is not especially limited, but it may be comprised in an amount of 0.01 to 20% by weight based on the total composition weight. When the active ingredient satisfies the above dose, it can exhibit excellent effect without side effects. The cosmetic composition may additionally comprise functional additives and ingredients which are included in general cosmetic compositions. The functional additives may include an ingredient selected from the group consisting of water-soluble vitamin, oil-soluble vitamin, polymer peptide, polymer polysaccharide, sphingolipid, and seaweed extracts. The cosmetic composition of the present invention may further comprise ingredients which are comprised in general cosmetic compositions as needed in addition to the functional additives. In addition to the ingredients, mixing ingredients include oil and fat ingredient, humectants, emollient, surfactant, organic and inorganic pigment, organic powder, ultraviolet absorber, antiseptic, bactericide, antioxidant, plant extract, pH, alcohol, pigment, spice, blood flow stimulant, cooling agent, antiperspirant, purified water, etc.

Since it is demonstrated that DHPV included in the composition according to the present invention is a metabolite of cacao which is a botanical raw material harmless to human body and is not only excellent in biosafety, but also regulates expression of proteins related to skin aging epigenetically, thereby exhibiting an excellent skin aging-preventing function at a low concentration of 1 nM based on skin cells, it is excellent in use as medicines, cosmetics or foods having high biostability.

As other aspect, the present invention relates to a composition for promoting skin elasticity, or preventing or improving skin wrinkles comprising DHPV (5-(3′,4′-dihydroxyphenyl)-R-valerolactone) as an active ingredient. Such composition may be a pharmaceutical composition, a cosmetic composition or a food composition, and components, dose and administration routes of the composition, preparation and content of DHPV which is an active ingredient, other additives and excipients, etc. are all applicable as same as the composition for preventing, improving or treating photoaging of skin.

### Advantageous Effects

Since DHPV, an active ingredient comprised in the composition according to the present invention is a metabolite of cacao which is a botanical raw material harmless to human body and is not only excellent in biosafety, but also regulates expression of proteins related to skin aging epigenetically, thereby exhibiting an excellent skin aging-preventing function at a low concentration of 1 nM based on skin cells, the composition comprising the same is excellent in use as medicines, cosmetics and foods for preventing, improving and treating skin aging.

### BRIEF DESCRIPTION OF DRAWINGS

FIG. 1 shows the synthesis process of DHPV.

FIG. 2 is the result of confirming expression regulation of one of aging markers of epidermal cells, Involucrin (IVL) and a novel aging marker PFN1 with western blot.

FIG. 3 is the result of confirming dermal fibroblast anti-aging function of DHPV. (A) shows the MMP-1 real time PCR result, and (B) shows AP-1 luciferase reporter gene assay result, respectively.

FIG. 4 shows the result that DHPV inhibits methylation in Involucrin (IVL) DNA promoter region induced by photoaging.

FIG. 5 shows the result of confirming biostability of DHPV.

### DETAILED DESCRIPTION OF THE INVENTION

Hereinafter, the configuration of the present invention will be described in more detail with reference to specific Examples. However, it will be apparent to one skilled in the art that the scope of the present invention is not limited by the description of Examples.

### Example 1: Confirmation of Aging-Preventing Effect of Skin Epidermal Cells by DHPV

In order to confirm skin photoaging-preventing effect by DHPV, the active ingredient of the composition according to the present invention, DHPV prepared by the following synthetic procedure was used in the Pharmacology Department of CHA University and tested as follows.

1-1. Synthesis of DHPV

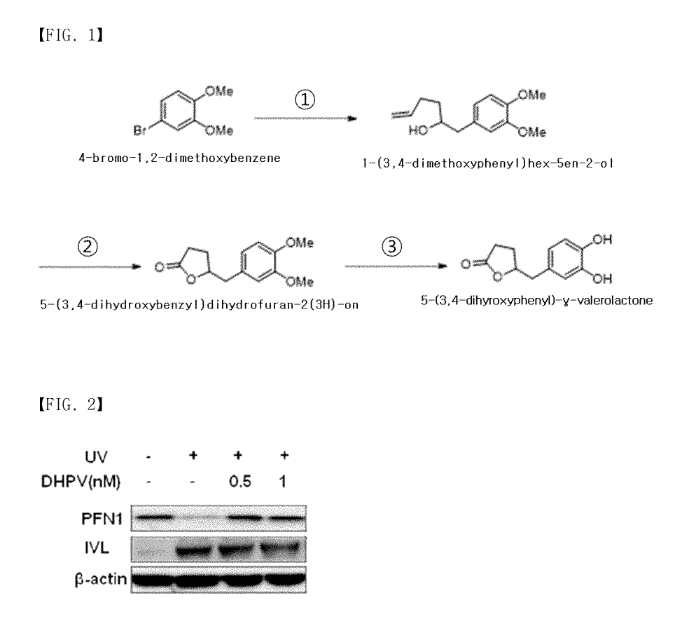

The synthesis of DHPV was conducted largely in three stages as shown in FIG. 1.

### (A) Preparation of 1-(3,4-dimethoxyphenyl) hex-5-en-2-ol)

4-Bromo-1,2-dimethoxybenzene (3.0 g, 13.8 mmol) was dissolved in anhydrous THF (15 ml) and cooled to −78° C., and then n-butyllithium (2.5 M, 6.1 ml, 15.2 mmol) was added drop by drop. After stirring for 40 min, BF3.OEt2 (1.9 ml, 15.2 mmol) and 2-(but-3-en-1-yl)oxirane (1.4 g, 13.8 mmol) were added. After stirring the mixture at the room temperature for 24 hrs and treating with water, aqueous layers were extracted with ethyl acetate. The organic layers were washed once with saturated NaHCO₃ solution, and dried with MgSO₄, and residues obtained by filtering and concentrating under reduced pressure were purified by silica gel elution chromatography to obtain a secondary alcohol. (yield 81%, 2.6 g)

### (B) Preparation of 5-(3,4-dimethoxybenzyl) dihydrofuran-2(3H)-one

The compound obtained in (A) of 1-1 (1.0 g, 4.2 mmol) was dissolved in 1,4-dioxane (4 ml) and water (1 ml), and 2,6-lutidine (0.97 ml, 8.4 mmol) and OsO₄ were added. After cooling by 0° C., sodium periodate (3.6 g, 16.8 mmol) was added. After stirring the mixture at the room temperature for 17 hrs and treating with water, aqueous layers were extracted with CH₂Cl₂. Organic layers were washed with salt water and dried with, and lactol compounds were obtained by filtering and concentrating under reduced pressure. The obtained lactol compounds were dissolved in CH₂Cl₂ (5 ml) and cooled by 0° C., and then sodium acetate (688 mg, 8.4 mmol) and pyridinium chlorochromate (1.8 g, 8.4 mmol) were slowly added. After stirring the mixture at the room temperature for 1 hr, lactone compounds were obtained by purification with silica gel elution chromatography. (yield 73%, 724 mg)

### (C) Preparation of 5-(3,4-dihydroxyphenyl)-Γ-valerolactone

The lactone compound obtained in (B) (500 mg, 2.1 mmol) was dissolved in CH₂Cl₂ (2 ml) and cooled by −78° C., and then boron tribromide (0.81 ml, 8.4 mmol) was added. After stirring the mixture at the room temperature for 10 hrs and treating with water, aqueous layers were extracted with CH₂Cl₂. The organic layers were washed with salt water, and dried with MgSO₄, and residues obtained by filtering and concentrating under reduced pressure were purified by silica gel elution chromatography to obtain white solid valerolactone. (yield 33%, 144 mg)

1-2. Confirmation of Skin Aging-Preventing Function of DHPV

(A) Confirmation of DHPV Effect on Human Derived Epithelial Cells

1) Photoaging Cell Induction

First, aging was induced by irradiation with 0.1 J/cm² UV on human skin cells (hypodermis and epidermis were separated using HaCaT cell line purchased from ATCC and human body derived Foreskin, and primary cell culture was used). Next, DHPV, a metabolite of cacao, prepared in 1-1 was treated 0.5, 1 nM, respectively, and cells were obtained.

2) Immunoblot

The expression level of IVL (involucrin) which is one of aging markers of epidermal cells was confirmed by Western blot. Cells were degraded using cell dissolution buffer containing protease inhibitor cocktail kit (P3100-005; GenDEPOT, Barker, Tex.), and phosphatase inhibitor (sc-45065; Santa Cruz Biotechnology, Santa Cruz, Calif.) (#9803; Cell SignalingTechnology, Danvers, Mass.). The aliquot containing 25 ug of cell lysates was separated by electrophoresis on SDS-polyacrylamide gel, and then transferred to nitrocellulose membrane (Amersham, Buckinghamshire, UK) for 180 min at 75 V and 40° C. on a transfer buffer (25 mM Tris, 192 mM glycine, 20% [v/v] methanol [pH 8.3]). The membrane was blocked with PBS of 3% (w/v) BSA containing 0.1% (v/v) Tween-20 for 60 min, and then cultured with anti-involucrin (Santacruz, sc28557) antibody. HRP-combined goat anti-rabbit IgG (1:1000; sc 2004, SantaCruz) was used as a secondary antibody. ECL lumi Femto solution A/B (Dogen, Seoul, Korea) was used to develop blots.

The expression level of PFN1 as a novel aging marker of epidermal cells was confirmed with Western blot by the same method as the above method except using anti-PFN1 (Abcam, ab118984) antibody instead of anti-involucrin (Santacruz, sc28557) antibody and HRP-combined goat anti-mouse IgG (1:3000; sc 2005, SantaCruz) instead of HRP-combined goat anti-rabbit IgG (1:1000; sc 2004, SantaCruz).

3) Result Interpretation

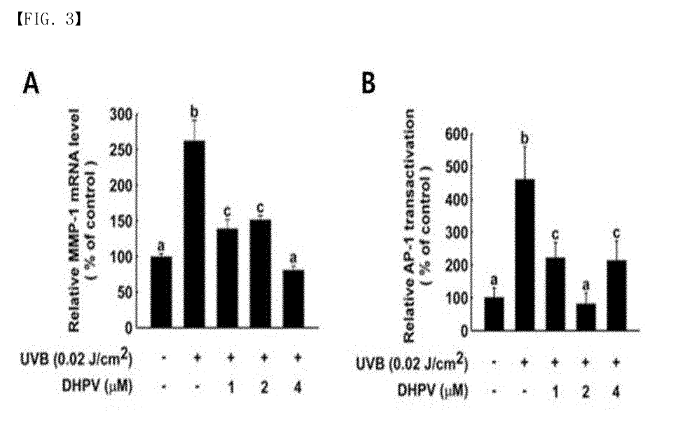

As shown in FIG. 2, it was demonstrated that the expression level of the IVL marker was increased when UV was irradiated, and it was demonstrated that the amount of increased IVL was reduced in a concentration-dependent manner when treating DHPV. In addition, it was demonstrated that the expression level of the PFN1 marker was reduced when UV was irradiated, and it was demonstrated that the amount of increased PFN1 was increased in a concentration-dependent manner when treating DHPV. This means that DHPV has a function to recover skin aged by UV.

(B) Confirmation of Anti-Aging Effect of DHPV in Hypodermal Cells

MMP-1 and AP-1 were skin aging markers whose expression and activity were increased when UVB irradiation, and the anti-aging effect of DHPV was investigated by changes of its expression level when treating DHPV.

1) Induction of Photoaging of Human Derived Hypodermis Fibroblasts

First, human derived hypodermis fibroblasts (hypodermis and epidermis were separated using human body derived Foreskin and the primary cell culture was used) were cultured in the DMEM medium containing 10% FBS (fetal bovine serum), and then cultured in serum free media for 24 hrs and UVB was irradiated. Next, cells were cultured in a medium containing serum or a medium containing 1-41M DHPV for 24 hrs, thereby collecting cells.

2) MMP-1 Real Time PCR

In order to confirm changes of mRNA expression level of MMP-1, mRNA was obtained using RNAiso Plus (Takara Bio Inc., Shiga, Japan). Next, CDNA was synthesized and prepared using PrimeScript™ 1^{st} Plus (Takara Bio Inc., Shiga, Japan), and real time PCR was conducted using IQ SYBR (Bio-Rad Laboratories). The primer sequences used in conducting PCR were as same as the following Table 1.

TABLE 1
real time PCR primer of photoaging-induced skin cells

| name | sequence (5′→3′) | SEQ ID NO. | Tm (° C.) |
|---|---|---|---|
| MMP-1 forward | CCC CAA AAG CGT GTG ACA GTA | 1 | 59.8 |
| MMP-1 reverse | GGT AGA AGG GAT TTG TGC G | 2 | 55.1 |
| GAPDH forward | GAG TCA ACG GAT TTG GTC GT | 3 | 57.3 |
| GAPDH reverse | TTG ATT TTG GAG GGA TCT CG | 4 | 54.2 |

3) AP-1 Luciferase Reporter Gene Assay

AP-1 was obtained by inoculating pGF-AP1-mCMV-EF1-Puro vector (System Biosciences, CA) and a package vector, pMD2.0G and psPAX (Addgene Inc, Cambridge, Mass.) to HEK293T cell and in 36 hrs, viral particles were obtained using 0.45-mm injection filter, in order to measure activity changes by UVB irradiation through luciferase reporter gene assay. Next, it was inoculated to hypodermis fibroblasts, and cells that injection with puromycin (Sigma, MO, 21 g/mL) was successes were obtained. Then the activity of AP-1 was measured by using luciferase assay kit (Promega, Madison, Wis.) after culturing cells in the same manner with the hypodermal fibroblast culturing method and treating with DHPV.

4) Result Interpretation

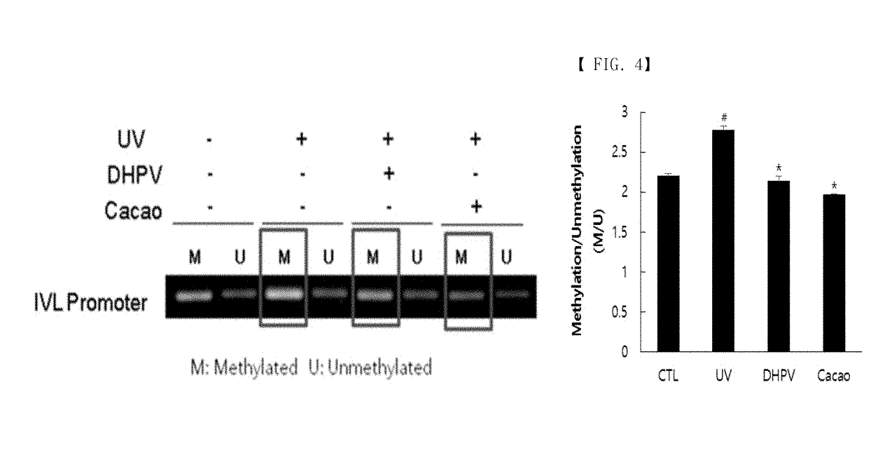

FIG. 3 is the experimental result to the experiment procedure, and it was demonstrated that the expression of MMP-1 and activity of AP-1 were increased approximately 2.5 fold more in case of MMP-1 and approximately 4.7 fold more in case of AP-1 than the control group when irradiating UVB. It was demonstrated that the mRNA expression of MMP-1 and activity of AP-1 were recovered similarly to the control group when treating DHPV since then. This is a result of confirming that DHPV functions not only in anti-aging function in epithelial cells identified in 1) but also in hypodermal cells. It was confirmed that DHPV was a substance having a skin anti-aging function through this.

(C) DNA Methylation Analysis

1) Analysis of Photoaging Cell Induction and IVL Gene Methylation

The aging was induced by irradiation with 0.1 J/cm² UV on human skin cells (hypodermis and epidermis were separated using HaCaT cell line purchased from ATCC and human body derived Foreskin, and primary cell culture was used). DNA of the cells was extracted using NucleoSin® Tissue Kit (Macherey-Nagel, Düren, Germany). The extracted DNA was quantified using Nano Drop and its quality was confirmed. In the extracted DNA, unmethylated Cytosine residues were converted to Uracil residues by treating Bisulfite using EZ DNA Methylation-Lightning™ Kit (Zymoresearch, CA, USA) for methylation analysis.

Distilled water was added to Bisulfited DNA 100 ng, primer pair (20 pmole) respectively, 1 uL, TaKaRa EpiTaq HS (5 U/μl) 0.25 uL, 10× EpiTaq PCR Buffer (Mg2⁺ free) 5 uL, 25 mM MgCl₂ 5 uL, dNTP Mixture (2.5 mM each) 6 uL to be 50 uL of the final volume, and then after reacting for 3 hrs at 98° C., the procedure of 10 sec at 98° C., 1 min at 55° C., and 30 sec at 72° C. as 40 cycles was repeated and reacted. Then, after extending for 5 min at 72° C., the result was analyzed in 2% (w/v) agarose gel.

2) Result Interpretation

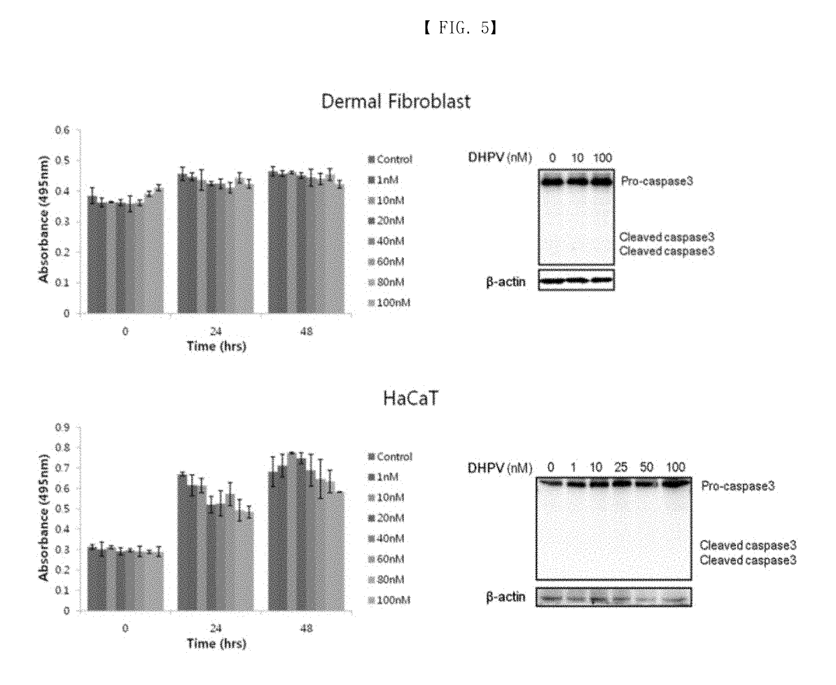

As shown in FIG. 4, it was demonstrated that DHPV inhibited methylation of Involucrin (IVL) DNA promoter region induced by photoaging. Methylation of IVL promoter by DHPV and relative methylation tendency were analyzed by methylation specific PCR which can relatively compare methylation or non-methylation of genes according to band intensity on the electrophoresis. As shown in FIG. 4, it was demonstrated that the intensity of gene methylation band of UV treated experimental group was strong compared to UV untreated control group, and it was demonstrated that the intensity of methylation band was decreased when treating DHPV. The methylation specific PCR may be quantified by comparing and analyzing the intensity of methylated gene band versus unmethylated gene band, and the intensity change of band shown by the electrophoresis result can be seen from the graph of FIG. 4.

### Example 2: Evaluation of DHPV Biostability

1) Evaluation of Biostability and Cell Preparation Therefor

In order to evaluate DHPV biostability, HaCaP cell line and hypodermal cells isolated from human derived foreskin were inoculated (2×10⁴ cell each) and cultured on the 24 well plate and stabilized. Then, the cells were cultured in serum free media for 24 hrs and 48 hrs. Next, cell viability thereof was analyzed by method suggested in Ez-cytox (DoGen, EZ-1000).

Further, HaCaP cell line and hypodermal cells were inoculated (1×10⁶ cell) and cultured on the 10 cm² culture dish. Then, the cells were cultured in serum free media for 24 hrs. Next, the cells were cultured in the same media with treatment of DHPV for 24 hrs, thereby collecting cells. Biostability was evaluated by immunoblot.

2) Immunoblot

The protein excision of caspase-3 protein which is one of apoptosis markers of cells was confirmed by Western blot. According to the results of Western blot, in case of caspase-3, 35 kDa of protein was excised into about 17 kDa of protein fragment by cell apoptosis.

Cells were degraded using cell dissolution buffer containing protease inhibitor cocktail kit (P3100-005; GenDEPOT, Barker, Tex.), and phosphatase inhibitor (sc-45065; Santa Cruz Biotechnology, Santa Cruz, Calif.) (#9803; Cell SignalingTechnology, Danvers, Mass.). The aliquot containing 25 ug of cell lysates was separated by electrophoresis on SDS-polyacrylamide gel, and then transferred to nitrocellulose membrane (Amersham, Buckinghamshire, UK) for 180 min at 75 V and 40° C. on a transfer buffer (25 mM Tris, 192 mM glycine, 20% [v/v] methanol [pH 8.3]). The membrane was blocked with PBS of 3% (w/v) BSA containing 0.1% (v/v) Tween-20 for 60 min, and then cultured with anti-caspase-3 (Cell Signaling, #9662) antibody. HRP-combined goat anti-rabbit IgG (1:1000; sc 2004, SantaCruz) was used as a secondary antibody. ECL lumi Femto solution A/B (Dogen, Seoul, Korea) was used to develop blots.

The expression level of PFN1 as a novel aging marker of epidermal cells was confirmed with Western blot by the same method as the above method except using anti-PFN1 (Abcam, ab118984) antibody instead of anti-involucrin (Santacruz, sc28557) antibody and HRP-combined goat anti-mouse IgG (1:3000; sc 2005, SantaCruz) instead of HRP-combined goat anti-rabbit IgG (1:1000; sc 2004, SantaCruz).

3) Result Interpretation

As shown in FIG. 5, it was demonstrated that the biostability of DHPV was shown similarly to that of control group. Also, similar result was shown in cell apoptosis through caspase-3. This means that in vivo toxicity of DHPV would not be incurred.

1. A method of promoting skin elasticity, preventing, treating or improving photoaging of skin, or preventing or improving skin wrinkles in a subject, the method comprising administering to a subject in need thereof a therapeutically effective amount of a composition including DHPV (5-(3′,4′-dihydroxyphenyl)-R-valerolactone) of the following chemical formula 1.

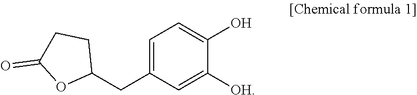 [SMILES: O=C1CCC(Cc2ccc(O)c(O)c2)O1]

2. The method of claim 1, wherein the DHPV of the chemical formula 1 epigenetically reduces expression of skin aging specific protein to prevent photoaging of skin, promote elasticity, or prevent or improve wrinkles.

3. The method of claim 1, wherein the DHPV of the chemical formula 1 is comprised in an amount of 0.1 to 99.9% by weight based on the total composition weight.

4. The method of claim 1, wherein the composition is a food composition in the form of functional food, health supplement food or health functional food.

5. The method of claim 1, wherein the composition is a pharmaceutical composition in oral or parenteral form.

6. The method of claim 1, wherein the composition is a cosmetic composition in the formulation of one or more selected from the group consisting of skin lotion, skin softener, skin toner, astringent, lotion, milk lotion, moisture lotion, nutrition lotion, massage cream, nutrition cream, moisture cream, hand cream, foundation, essence, nutrition essence, pack, soap, cleansing foam, cleansing lotion, cleansing cream, body lotion and body cleanser.
